(19) United States
(12) Patent Application Publication
(10) Pub. No.: US 2006/0019320 A1
(43) Pub. Date: Jan. 26, 2006

(54) Wnt mediated erbb signalling, compositions and uses related thereto

(76) Inventors: Gianluca Civenni, Basel (CH); Thomas Holbro, Basel (CH); Nancy Hynes, Basel (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/528,439

(22) PCT Filed: Sep. 19, 2003
(86) PCT No.: PCT/EP03/10469

§ 371 Date: Mar. 18, 2005

(30) Foreign Application Priority Data
Sep. 20, 2002 (GB) 0221952.5

(51) Int. Cl. G01N 33/574; A61K 38/17

(52) U.S. Cl. 435/7.23; 514/12

(57) ABSTRACT

The present invention makes available assays and reagents inhibiting ErbB signals produced by Wnt protein comprising contacting a cell expressing ErbB and Wnt receptor (Frizzled) with a Wnt antagonist in a sufficient amount to reduce the sensitivity of the cell to ErbB signaling.

### FIELD OF THE INVENTION

The present invention relates to ErbB signalling, in particular to methods of modulating ErbB signalling using Wnt antagonists or agonists and to methods of screening for agents effective in modulating ErbB signalling. The methods are useful in developing pharmaceuticals, in particular for the treatment of cancer and other diseases or conditions dependent on ErbB signalling.

### BACKGROUND OF THE INVENTION

Wnts are a large family of secreted glycoproteins that play an important role in normal development. The mammary gland expresses multiple Wnts (Gavin and McMahon, 1992), and some, like Wnt4, have been shown to have specific developmental roles (Brisken et al., 2000). Wnt1, was a prototypic oncogene first detected in mouse mammary tumor virus (MMTV)-induced mammary cancer (Nusse and Varmus, 1982) and Wnt5a is normally expressed in the mammary gland (Gavin and McMahon, 1992). At least ten Wnt genes have been identified in the mouse (Wnt-1, 2, 3, 3a, 4, 5a, 5b, 6, 7a, and 7b; Gavin et al., (1990), Genes Dev., 4, pp. 2319-2332) and seven Wnt genes have been identified in the human (Wnt-1, 2, 3, 4, 5a, 7a, and 7b) by cDNA cloning (Vant Veer et al., 1984, Mol. Cell. Biol., 4, pp. 2532-2534; Wainright et al., 1988, EMBO J., 7, pp. 1743-1748). See also http://www.stanford.edu/˜rnusse/wntwindow.html, U.S. Pat. Nos. 5,780,291; 6,043,053; 6,100,060 and 6,297,030.

Members of the Frizzled (Fz) family of seven-pass transmembrane proteins are receptors for Wnt proteins (Wang et al. J Biol Chem Feb. 23, 1996;271 (8):4468-76; http://www.stanford.edu/˜rnusse/wntwindow.html). Wnt binding to Fz initiates a pathway that prevents glycogen synthase kinase-3B (GSK-3B) from phosphorylating beta-catenin, one of its critical substrates. This leads to beta-catenin stabilization and translocation to the nucleus where it engages transcription factors of the TCF (T-cell factor) family (van Noort and Clevers, 2002; Nusse, 1999). This pathway is a driving force in development of some human cancers, such as colon cancer and melanomas (Polakis, 2000; Bienz and Clevers, 2000).

The ErbB family of receptors and their activating ligands, the EGF-related peptides, have important functions in the normal mammary gland (Troyer and Lee, 2001) and in breast cancer (Olayioye et al., 2000). Furthermore, ErbB1 has emerged as an important mediator of signaling from other classes of membrane receptors including: G protein coupled receptors (GPCRs), other receptor tyrosine kinases (RTKs), cytokine receptors and integrin receptors (Carpenter, 1999; Gschwind et al., 2001).

A need exists to find components that are involved in or that affect signalling pathways to allow more accurate and effective diagnosis and treatment of diseases dependent on these signalling pathways, such as cancer. A more complete delineation of the ErbB1 signalling pathway and identification of the pathway's components provided by the present invention meets this need.

### RELEVANT LITERATURE

Bienz, M. (1998) TCF: transcriptional activator or repressor? Curr. Opin. Cell Biol., 10, 366-372.

Bienz, M. and Clevers, H. (2000) Linking colorectal cancer to Wnt signaling. Cell, 103, 311-320.

Brandt, R., Wong, A. M. L. and Hynes, N. E. (2001) Mammary glands reconstituted with Neu/ErbB2 transformed HC11 cells provide a novel orthotopic model for testing anti-cancer agents. Oncogene, 20, 5459-5465.

Brantjes, H., Roose, J., van de Wetering, M. and Clevers, H. (2001) All Tcf HMG box transcription factors interact with groucho-related co-repressors. Nucl. Acids Res., 29, 1410-1419.

Brisken, C. et al., (2000) Essential function of Wnt-4 in mammary gland development downstream of progesterone signaling. Genes Develop., 14, 650-654.

Carpenter, G. (1999) Employment of the epidermal growth factor receptor in growth factor-independent signaling pathways. J. Cell Biol., 146, 697-702.

Dong, J., Opresko, L. K., Dempsey, P. J., Lauffenburger, D. A., Coffey, R. J. and Wiley, H. S. (1999) Metalloprotease-mediated ligand release regulates autocrine signaling through the epidermal growth factor receptor. Proc. Natl. Acad. Sci. USA, 96, 6235-6240.

Gavin, B. and McMahon, A. P. (1992) Differntial regulation of the wnt gene family during pregnancy and lactation suggests a role in postnatal development of the mammary gland. Mol. Cell. Biol., 12, 2418-2423.

Gschwind, A., Zwick, E., Prenzel, N., Leserer, M. and Ullrich, A. (2001) Cell communication networks: epidermal growth factor receptor transactivation as the paradigm for interreceptor signaln transmission. Oncogene, 20, 1594-1600.

He, T. C. et al., (1998) Identification of c-Myc as a target of the APC pathway. Science, 281, 1509-1512.

Hynes, N. E., et al, (1990) EGF receptor, but not c-erbB2, activation prevents lactogenic hormone induction of the b-casein gene in mouse mammary epithelial cells. Mol. Cell.

Biol., 10, 4027-4034.

Lane, H. A., Beuvink, I., Motoyama, A. B., Daly, J. M., Neve, R. M. and Hynes, N. E. (2000) ErbB2 potentiates breast tumor proliferation through modulation of p27(Kip1)-Cdk2 complex formation: receptor overexpression does not determine growth dependency.

Mol. Cell. Biol., 20, 3210-3223.

Massague, J. and Pandielia, A. (1993) Membrane-anchored growth factors. Annu. Rev.

Biochem., 62, 515-541.

Mitamura, T. et al., (1997) Structure-function analysis of the diptheria toxin receptor toxin binding site by site-directed mutagenesis. J. Biol. Chem., 272, 27084-27090.

Neve, R., et al., (2000) Effects of oncogenic ErbB2 on G1 cell cycle regulators in breast tumor cells. Oncogene, 19, 1647-1656.

Nusse, R. (1999) Wnt targets. Repression and activation. Trends Genet., 15, 1-3.

Nusse, R. and Varmus, H. E. (1982) Many tumors induced by the mouse mammary tumor virus contain a provirus integrated in the same region of the host genome. Cell, 31, 99-109.

Olayioye, M. A., Neve, R. M., Lane, H. A. and Hynes, N. E. (2000) The ErbB signaling network: receptor heterodimerization in development and cancer. EMBO J., 19, 3159-3167.

Polakis, P. (2000) Wnt signaling and cancer. Genes Dev., 14, 1837-1851.

Prenzel, N., et al. (1999) EGF receptor transactvation by G-protein=coupled receptors requires metalloproteinase cleavage of proHB-EGF. Nature, 402, 884-888.

Sorensen, B. S., Torring, N., Bor, M. V. and Nexo E. (2000) Quantitation of the mRNA expression of the EGF system: selective induction of HB-EGF and AR expression by growth factor stimulation of prostate stromal cells. J. Lab. Clin. Med., 136, 209-217.

Tavema, D., Groner, B. and Hynes, N. E. (1991) EGF receptor, PDGR receptor, and c-erbB2 receptor activation all promote growth but have distinctive effects upon mouse mammary epithelial cell differentiation. Cell Growth Differ., 2,145-154.

Tetsu, O. and McCormick, F. (1999) Beta-catenin regulates expression of cyclinD1 in colon carcinoma cells. Nature, 398, 422-426.

Traxler, P., et al., (2001) Tyrosine kinase inhibitors: from rational design to clinical trials. Med. Res. Rev., 21, 499-512.

Troyer, K. L. and Lee, D. C. (2001) Regulation of mouse mammary gland development and tumorigenesis by the ErbB signaling network. J Mammary Gland Biol. Neoplasia, 6, 7-21.

Ugolini, F. et al., (2001) WNT pathway and mammary carcinogenesis: loss of expression of candidate tumor suppressor gene SFRP1 in most invasive carcinomas of the medullary type. Oncogene, 20, 5810-5817.

Uren, A., et al., (2000) Secreted frizzled-related protein 1 binds directly to wingless and is a biphasic modulator of Wnt signaling. J. Biol. Chem., 275, 4374-4382.

Van Noort, M. and Clevers, H. (2002) TCF transcription factors, mediators of Wnt-signaling in development and cancer. Dev. Biol., 244,1-8.

Wang, Y., Johnson, A. R., Ye, Q. Z. and Dyer, R. D. (1999) Catalytic activities and substrate specificity of the human membrane type 4 matrix metalloproteinase catalytic domain. J. Biol. Chem., 274, 33043-33049.

Willert, K, Brown, J. D., Danenberg, E., Duncan, A. W., Weissmann, I. L., Reya, T., Yates, J. R. 3rd and Nusse, R. (2003) Wnt proteins are lipid-modified and can act as stem cell growth factors. Nature, 423, 448-452.

Yu, Q., Geng, Y. and Sicinski, P. (2001) Specific protection against breast cancers by cyclin D1 ablation. Nature, 411, 1017-1021.

### SUMMARY OF THE INVENTION

In one aspect the invention provides methods for modulating ErbB receptor signalling, comprising contacting a cell expressing ErbB receptors and Frizzled (Fz) at the cell surface with a Wnt agonist or antagonist in a sufficient amount to affect ErbB receptor signalling of the cell. In one embodiment a Wnt antagonist is used to inhibit (reduce) ErbB receptor signalling. In another embodiment, a Wnt agonist is used to increase ErbB signalling. The ErbB receptor can comprise one or more of ErbB1, ErbB2, ErbB3 and ErbB4. Thus, the cell could be a cancer cell, in particular a breast cancer or colon cancer cell. In some embodiments, the Wnt antagonist is an antagonist of Wnt1 or Wnt5a. In particular, the Wnt antagonist can be an antibody or fragment thereof, which specifically binds to Wnt or its receptor, Fz, secreted Frizzled related protein (sFRP) or a small molecule. Thus, also provided is the use of a Wnt antagonist to inhibit ErbB receptor signalling.

In a further aspect, the present invention provides a method of screening for compounds effective in modulating Wnt-mediated ErbB receptor signalling, comprising: contacting a Wnt receptor (e.g., Fz) with Wnt in the presence of a candidate compound, detecting binding of Wnt or the candidate compound to the Wnt receptor and correlating the binding of the candidate compound to the Wnt receptor or a change in binding of Wnt to the Wnt receptor relative to when the candidate compound is absent with a potential ErbB modulator. In preferred embodiments, the method further comprises determining ErbB signalling, such as by the presence of ERK activity, MAPK activity, ErbB phosphotyrosine or cyclin D. Thus, cell base assays are preferred. In an alternative embodiment, ErbB signalling is detected by the presence of a reporter gene product. The screening methods of the invention can be used to identify candidate compounds that inhibit ErbB signalling (Wnt antagonists) as well as candidate compounds that increase ErbB signalling (Wnt agonists).

Also provided by the invention are kits comprising: a Wnt, a Fz, and/or a cell expressing Wnt and/or Fz; and a means of detecting ErbB signalling, such as an antibody, optionally comprising a detectable tag or label.

In a further aspect of the invention, a method for inhibiting ErbB signalling in a patient is provided, the method comprising administering to the patient a composition comprising a Wnt antagonist in a sufficient amount to reduce the ErbB signalling in a cell of the patient. The antagonist is an antibody or a fragment thereof that specifically binds to Wnt or Fz. Such methods can be used to treat diseases or conditions dependent on ErbB signalling, such as cancer, in particular cancers expressing ErbB 1, such as certain breast or colon cancers.

Thus, also provided by the invention is the use of a Wnt antagonist as a pharmaceutical for the treatment of ErbB expressing cancers (or other diseases or conditions dependent on ErbB signalling), the use of a Wnt antagonist in the manufacture of a medicament for the treament of ErbB expressing cancers, a Wnt antagonist for the treatment of ErbB expressing cancers, as well as compositions comprising a Wnt antagonist and a pharmaceuticaly acceptable carrier for the treatment of ErbB expressing cancers. Conversely, Wnt agonists can replace Wnt antagonist depending on the indication.

In a further aspect of the invention, a method of diagnosing a patient in need of treatment with a Wnt antagonist is provided, the method comprising detecting altered ErbB receptor signalling in a sample compared to a control sample.

### DETAILED DESCRIPTION OF THE INVENTION

For convience, certain terms employed in the specfication, examples, and appended claims are set out below.

The term “Wnt” encompasses preparations of Wnt polypeptides and peptidyl fragments thereof, including without limitation Wnt-1, 2, 3, 3a, 4, 5a, 5b, 6, 7a, and 7b and Wnt x.

The term “Wnt receptor” is meant to include Frizzled (Fz) as well as fragments and variants thereof.

The term “variant” is meant to include polypeptides having an altered amino acid sequence, which may be in the form of a fusion with another protein sequence, for example, tags for the targeted delivery or detection. The variant may include modified peptide linkages or non-naturally occurring amino acids, which may have Improved properties such as stability or activity are included. A “variant” in terms of amino acid sequence defines an amino acid sequence that differs by one or more amino acids from another, usually related amino acid sequence. The variant may have “conservative” changes, wherein a substituted amino acid has similar structural or chemical properties (e.g. replacement of leucine with isoleucine). Less likely, a variant may have “non-conservative” changes, e.g. replacement of a glycine with a tryptophan. Similar minor variations may also include amino acid deletions or insertions (i.e. additions), or both. Guidance in determining which and how many amino acid residues may be substituted, inserted or deleted without abolishing activity may be found using computer programs well known in the art.

As used herein, “ErbB receptors” refers to receptor proteins comprising one or more ErbB subunits, such as ErbB1 (also known as EGF receptor), ErbB2, ErbB3 and ErbB4. As exemplified below, the biological activity of ErbB receptors is readily determined. For example, transactivation of ErbB1 receptors has a specific, measurable biological effect, namely stimulation of cyclin D1 expression through induction of ERK and/or MAPK signalling. As is apparent to one of ordinary skill in the art, other ErbB receptors or combinations thereof can induce other characteristic effects, such as inducing other signalling pathways (e.g., PI3K pathway).

The term “agonist”, with respect to Wnt, refers to a compound that mimics the action of a Wnt protein in ErbB receptor signalling.

The term “antagonist”, with respect to Wnt bioactivity, refers to a compound that inhibits Wnt-mediated ErbB receptor signal transduction. In the context of the present invention, such antagonists can include compounds with the ability to bind to a Wnt and block binding of Wnt to its receptor (Fz), compounds with the ability to bind to Fz and inhibit the simultaneous binding of Wnt to Fz, or, compounds with the ability to act in a non-competitive, allosteric and/or other similar manner, and thereby inhibit the response of an ErbB receptor to Wnt, provided that the compound affects metalloproteinase activity without inhibiting metalloproteinase (e.g., matrix metalloproteinase, MMP) directly.

The term “competitive antagonist” refers to a compound that binds to a receptor (Fz) site; and its effects can be overcome by increased concentration of the agonist (Wnt or Wnt-like activity).

An “effective amount” of, e.g., a Wnt antagonist, refers to an amount of the antagonist that brings about the desired decrease in ErbB receptor signalling, such as, a decrease in the rate of cell proliferation. Similarly, an “effective amount” of a Wnt agonist refers to an amount of the agonist that brings about the desired increase in ErbB receptor signalling.

The “growth state” of a cell refers to the rate of proliferation of the cell and/or the state of differentiation of the cell.

As used herein, “proliferating” and “proliferation” refer to cells undergoing mitosis.

The present invention relates to the discovery that signal transduction pathways dependent on ErbB receptors can be activated by Wnt. As set out in more detail below, the present inventors demonstrate for the first time, that Wnt peptides transactivate ErbB1, which in turn leads to strong stimulation of the MAPK pathway. While not wishing to be bound by any particular theory, Wnt presumably stimulates the MAPK pathway by increasing the availability of ErbB1 ligands (or other ligands that bind to ErbB receptors). Therefore, antagonists of Wnt interfere with the activity of the ErbB receptor and have potential therapeutic applications in various diseases, including for example, cancer.

Modulation of Wnt-Mediated ErbB Receptor Signalling

Thus, one aspect of the invention relates to methods for inhibiting ErbB receptor signalling, comprising contacting a cell expressing ErbB receptors and Frizzled (Fz) at the cell surface with a Wnt antagonist in a sufficient amount to reduce the ErbB receptor signalling of the cell. The ErbB receptor typically comprises one or more of ErbB1, ErbB2, ErbB3 and ErbB4. In preferred embodiments, the Wnt antagonist is an antagonist of Wnt1 or Wnt5a and the ErbB receptor ErbB1.

The Wnt antagonist can be an antibody or fragments or variants thereof, which interfere with Wnt function in ErbB signalling. Such antibodies may specifically react with Wnt protein or its receptors (Fz). Antibodies generated against Wnt or Fz polypeptides can be obtained by administering the polypeptides or epitope-bearing fragments, variants or cells to an animal, preferably a nonhuman, using routine protocols. For preparation of monoclonal antibodies, any technique which provides antibodies from continuous cell line cultures can be used. Examples include the hybridoma technique (Kohler, G. and Milstein, C., Nature (1975) 256:495-497), the trioma technique, the human B-cell hybridoma technique (Kozbor et al., Immunology Today (1983) 4:72) and the EBV-hybridoma technique (Cole et al., Monoclonal Antibodies and Cancer Therapy, pp. 77-96, Alan R. Liss, Inc., 1985). Techniques for the production of single chain antibodies (U.S. Pat. No. 4,946,778) can also be adapted to produce single chain antibodies to polypeptides of this invention. Also, transgenic mice, or other organisms including other mammals, may be used to express humanized antibodies.

One aspect of the present invention relates to the use of sFRP, fragments or variants thereof, to interfere with ErbB receptor signalling produced by the Wnt proteins. Such a Wnt antagonist is illustrated below in Example 4. The secreted Frizzled related protein (sFRP) is shown to inhibit Wnt signalling through ErbB1 and therefore acts as an antagonist. Variants or fragments of sFRP maintain their ability to inhibit Wnt signalling to interfere with ErbB receptor signalling.

It is also specifically contemplated that small molecules, which similarly intefere with Wnt dependent aspects of ErbB receptor activity will likewise be capable of inhibiting ErbB receptor-mediated signals. In preferred embodiments, the subject inhibitors are organic molecules having a molecular weight less than 2500 amu, more preferably less than 1500 amu, and even more preferably less than 750 amu, and are capable of inhibiting at least some of the biological activities of ErbB receptor signalling that are dependent on Wnt.

The methods of the present invention include the use of Wnt antagonists to inhibit ErbB receptor signalling in a wide range of cells, tissues and organs expressing ErbB receptors and receptors for Wnt. The cells, tissue or organ will preferably express ErbB1, as well as Fz, in particular Fz 6, 7 and/or 8. Moreover, the subject methods can be performed on cells, which are provided in culture (in vitro), or on cells in a whole animal (in vivo).

Suitable cells will typically be any cell expressing ErbB receptors and Fz (or Wnt receptors), including without limitation epithelial cells or cells derived from epithelia, muscle cells, mesenchymal cells, such as glial or glioblastoma-cells, or cells derived from the neural crest, such as melanocytes and melanoma cells. The Examples below illustrate the Invention using mammary cells and human embryonic kidney cells. Cells can be placed into any known culture medium capable of supporting cell growth, including MEM, DMEM, RPMI, F-12 and the like, containing supplements which are required for cellular metabolism such as glutamine and other amino acids, vitamins, minerals and useful proteins such as transferrin and the like. Medium may also contain antibiotics to prevent contamination with yeast, bacteria and fungi such as penicillin, streptomycin, gentamicin and the like. In some cases, the medium may contain serum derived from bovine, equine, chicken and the like. The medium may also contain other biologically active molecules, such as growth factors, in particular EGF.

Conditions for culturing should be close to physiological conditions. The pH of the culture media should be close to physiological pH, preferably between pH 6-8, more preferably close to pH 7, even more particularly about pH 7.4. Cells should be cultured at a temperature close to physiological temperature, preferably between 30 C-40 C, more preferably between 32 C and 38 C, and most preferably between 35 C and 37 C.

Another aspect of the present invention relates to a method of modulating a differentiated state, survival, and/or proliferation of a cell expressing ErbB receptors and Fz, by contacting the cells with a Wnt antagonist according to the subject method and as the circumstances may warrant. For instance, it is contemplated by the invention that, in light of an apparently broad involvement of ErbB in various cell types and tissues in vertebrates, the subject method could be used as part of a process for modulating ErbB function in such tissues both in vitro and in vivo. The Wnt antagonist, whether inductive or anti-inductive with respect to proliferation or differentiation of a given cell or tissue, can be, as appropriate, any of the preparations described above, including antibodies, sFRP and small molecules.

Screening Assays

Also provided by the invention are methods of screening for compounds effective in modulating wnt-mediated ErbB receptor signalling. There are a variety of assays available for determining the ability of a compound to modulate ErbB signaling, many of which are readily amenable to high throughput formats. In many drug-screening programs that test libraries of compounds and natural extracts, high throughput assays are desirable in order to maximize the number of compounds surveyed in a given period of time. Thus, libraries of synthetic and natural products can be sampled for Wnt antagonists that inhibit ErbB signalling.

The availability of purified and recombinant Wnt, Fz and ErbB polypeptides, as well as cells expressing these polypeptides, facilitates the generation of assay systems which can be used to screen for drugs, such as small organic molecules, which are antagonists of the normal cellular function of Wnt, in particular its role in ErbB signalling. In one embodiment, the assay evaluates the ability of a compound to modulate binding between Wnt and a Wnt receptor (e.g., Fz) or merely direct binding to a Wnt receptor. In preferred embodiments, the assay scores for the ability of a test compound to alter Wnt-mediated ErbB signal transduction. In this manner, a variety of antagonists can be identified. A variety of assay formats will suffice and, in light of the present disclosure, will be comprehended by the skilled artisan.

Cell-free assays, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in an in vitro system, the assay instead being focused primarily on the effect of the drug on the molecular target as may be manifest in an alteration of binding affinity with receptor proteins.

Detection and quantification of Wnt-or candidate compound-Wnt receptor complexes provides a means for determining the test compound's efficacy at inhibiting (or potentiating) complex formation between Wnt and the Wnt receptor protein and affecting ErbB signalling. The efficacy of the compound can be assessed by generating dose response curves using various concentrations of the test compound. Moreover, a control assay can also be performed to provide a baseline for comparison. In the control assay, Wnt is added to the Wnt receptor protein, and the formation of Wnt/receptor complex is quantitated in the absence of the test compound. Alternatively, direct binding of the Wnt antagonist to the Wnt receptor can be detected and quantitated.

The Wnt receptor can be provided in the screening assay as a whole protein (preferably expressed on the surface of a cell), or alternatively as a fragment of the full length protein, which includes at least a portion which binds to Wnt, e.g. the extracellular domain. The Wnt receptor protein can be derived from a recombinant gene, e.g., being ectopically expressed In a heterologous cell. For instance, the protein can be expressed on mammalian cells (e.g., HC11, HEK293, COS, CHO, 3T3 or the like), or yeast cells by standard recombinant DNA techniques or can be on a cell that naturally expresses the receptor. These cells can be used for receptor binding, signal transduction or gene expression assays.

Complex formation between Wnt/a candidate compound and a Wnt receptor may be detected by a variety of techniques including without limitation by immunoassay, or by chromatographic detection. One Wnt binding assay is described in Bhanot, Nature 382:225(1996), for example. Modulation of the formation of complexes can be quantitated using, for example, detectably labelled proteins, such as radiolabelled, fluorescently labelled, or enzymatically labelled Wnt.

Typically, for cell-free assays, it will be desirable to immobilize either Wnt or the Wnt receptor to facilitate separation of receptor complexes from uncomplexed forms of one of the proteins (and/or antagonist), as well as to accommodate automation of the assay. In one embodiment, a fusion protein can be provided which adds a domain that allows the protein to be bound to a matrix. For example, glutathione-S-transferase/receptor (GST/receptor) fusion proteins can be adsorbed onto glutathione sepharose beads or glutathione derivatized microtitre plates, which are then combined with the binding partner (or potential binding partner), e.g. a labelled Wnt, and the test compound and incubated under conditions conducive to complex formation, e.g. at physiological conditions for salt and pH, though slightly more stringent conditions may be desired. Similarly, a (histidine)₆ tag can be used for immobilization through binding to nickel, or an epitope (e.g., HA tag) used for immobilization using an antibody. Following incubation, the beads (or other surface) are washed to remove any unbound ligand, and the matrix bead-bound label determined directly (e.g. beads placed in scintillant for a radiolabel), or in the supematant after the complexes are dissociated. Alternatively, the complexes can be dissociated from the bead and quantitated from using standard techniques. In some embodiments, both Wnt and Wnt receptor can be labelled and the candidate compound interferes with the binding of Wnt to a Wnt receptor to produce a detectable signal (e.g., fluorescence quenching by close proximity of Wnt to Fz produces fluorescence if a compound releases Wnt from the Wnt receptor).

In addition to cell-free assays, such as described above, the compounds of the subject invention can also be tested in cell-based assays, where the effect on ErbB receptor signalling can be assayed. In one embodiment, cells that are sensitive to Wnt induction, e.g. Fz-expressing cells or other cells sensitive to Wnt induction, can be contacted with a Wnt and/or a candidate compound of interest, with the assay scoring for anything from simple binding to the cell to inhibition in Wnt inductive responses by the target cell, in particular in ErbB signalling.

In general, such screening procedures can also involve producing appropriate cells that express the Wnt receptor and components of the ErbB signalling pathway. Such cells include cells from mammals, yeast, Drosophila or E. coli Cells expressing the receptor (or cell membrane containing the expressed receptor) are then contacted with a candidate compound to observe binding, or stimulation or inhibition of a functional response. Thus, the Fz-expressing cells can be cells that naturally express Fz protein (typically mammalian cells) or cells that have been genetically engineered to ectopically express Fz. Other characteristics of the cells may also be desired (e.g., ability for ErbB signalling when the assay is based on ErbB signalling). Furthermore, the recombinant cells can be engineered to include other heterolgous genes encoding proteins involved in Wnt-and/or ErbB-dependent signal pathways to design assays being sensitive to the functional reconstituion of the Wnt- and/or ErbB-signal transduction cascade.

The Wnt protein used in these cell-based assays can be provided as a purified source (natural or recombinant in origin), or in the form of cells/tissue which express the protein and which can be the target cells or cells co-cultured with the target cells.

Binding of a candidate compound to cells bearing the Wnt receptor can be detected by means of a label directly or indirectly associated with the candidate compound or in an assay involving competition with a labeled competitor. Inhibitors of activation are generally assayed in the presence of a known agonist (e.g., Wnt) and the effect on activation by the agonist by the presence of the candidate compound is observed. The values would typically be scored against a similar assay carried out in the absence of the test agent.

In addition to binding studies, by detecting changes in intracellular signals, such as alterations in second messengers (e.g., signal transduction, pH changes, or changes in calcium level), or gene expression in Fz- and ErbB-expressing cells contacted with a test agent, candidate Wnt antagonists affecting ErbB signalling can be identified. A number of gene products are implicated in Wnt-mediated ErbB receptor signalling, as is illustrated in the Examples below. For example, any one or combination of phosphotyrosine levels of ErbB1, MAPkinase activity, ERK activity or cyclin D1 levels can be assayed to determine ErbB1 transactivation. Similarly, depending on cell type or ErbB isoform, other pathways, such as the PI3K/PKB pathway may be modulated. If a potential Wnt antagonist (or agonist) binds the Wnt receptor (Fz), and thus inhibits (or enhances) Wnt binding to its receptor, the levels of Wnt-mediated ErbB activity will be reduced (or increased).

The induction of cells by Wnt proteins sets in motion a cascade involving the activation and inhibition of downstream effectors, the ultimate consequence of which is, in some instances, a detectable change in the transcription or translation of a gene. A transcriptional target of Wnt-mediated ErbB1 signaling is cyclin D1. By selecting transcriptional regulatory sequences from such a target gene that is up- or down regulated in response to ErbB signalling, and operatively linking such a promoter to a reporter gene, one can derive a transcription based assay which is sensitive to the ability of a specific test compound to modify Wnt-mediated ErbB signalling pathways. Expression of the reporter gene, thus, provides a valuable screening tool for the development of compounds that act as antagonists of Wnt in ErbB signalling. Reporter gene based assays of this invention measure the end stage of the Wnt-mediated ErbB cascade of events. Accordingly, in practicing one embodiment of the assay, a reporter gene construct is inserted into a cell in order to generate a detection signal dependent on Wnt signaling. The reporter gene may be detected at the mRNA level, e.g., using PCR, or at the protein level, for example a characteristic intrinsic activity (e.g., enzymatic activity, fluorescence, antibody reactivity). The amount of expression from the reporter gene is then compared to a control, suh as the amount of expression in the same cell in the absence of the test compound. Any statistically or otherwise significant difference in the amount of transcription indicates that the test compound has in some manner altered the signal transduction activity of the Wnt protein, e.g., the test compound is a potential Wnt antagonist/agonist.

To ensure that the candidate compound affects Wnt-mediated ErbB signalling, in addition to assaying ErbB transactivation, the candidiate compound will typically be screened for Wnt activity (e.g., binding to a Wnt receptor, downstream effects prior to modulation of metalloproteinase activity or downstream effects in a parallel pathway independent of metalloproteinase modulation, such as TCF induction). Metalloproteinase activity potentially stimulates the proteolytic cleavage and release of membrane-bound ErbB ligands. Thus, the Wnt antagonist does not inhibit metalloproteinase directly.

Although much of the discussion above relates to Wnt antagonists, it will be apparent to one of ordinary skill in the art in light of the present disclosure that Wnt agonists may also be identified by the screening methods of the invention. Similarly Wnt agonists can be used to activate ErbB signalling, in a similar manner to how Wnt antagonists are used to inhibit ErbB signalling. Thus, the methods can be used to determine whether the candidate compound activates or inhibits Wnt receptor binding or Wnt-mediated ErbB signalling and agonists are potentially useful in activating ErbB signalling when desired.

Compounds identified by the screening methods of the invention are amenable to combinatorial chemistry and other parallel synthesis schemes. The result is that large libraries of related compounds can be screened rapidly in high throughput assays in order to identify potential Wnt antagonist lead compounds, as well as to refine the specificity, toxicity, and/or cytotoxic-kinetic profile of a lead compound. For instance, Wnt bioactivity assays with respect to ErbB signalling as described above can be used to screen a combinatorial library for those having antagonist activity toward all or a particular ErbB/Fz isoform or activity.

In another aspect, the present invention relates to a kit for identifying agonists or antagonists of Wnt-mediated ErbB signalling, which comprises: a Wnt, a Fz, an ErbB receptor and/or a cell expressing Wnt, Fz and/or ErbB, and an antibody to or another means of detecting Wnt, Fz and/or ErbB signalling.

Therapeutic and Diagnostic Applications

In a further aspect the invention provides a method for inhibiting ErbB receptor signalling in a patient diagnosed with a condition or disasese dependent on altered ErbB signalling, comprising administering to the patient a composition comprising a Wnt antagonist (or agonist) in a sufficient amount to reduce (or increase) the ErbB receptor signalling in a cell of the patient. The antagonist can be an antibody that specifically binds to Wnt or Fz, for example, or a compound identified by the screening methods of the invention.

Thus, the identification of Wnt antagonists or agonists as modulators of ErbB receptor activity is useful in treating disease states involving ErbB receptor activity. Diseases and conditions dependent on altered ErbB signalling include disorders in cell growth and differentiation. Therefore, the Wnt antagonists and agonists have wide-ranging therapeutic applications including in the treatment of cancers (in particular solid tumors, e.g., breast cancer, colon cancer, prostate cancer, lung cancer, pancreatic cancer, lung cancer, glioblastoma, melanomas), cardiac disease, pancreatic disorders (e.g., Insulin production) and neurodegenerative diseases (such as Alzheimer's and Parkinson's diseases).

The subject method therefore has wide applicability to the treatment or prophylaxis of disorders afflicting a variety of cell types and tissues, as well as in cosmetic uses. In general, the method can be characterized as including a step of administering to an animal an amount of a Wnt antagonist (or agonist) effective to alter the growth state of a treated tissue. The mode of administration and dosage regimens will vary depending on the tissue(s), which is to be treated. For example, topical formulations will be preferred where the treated tissue is epidermal tissue, such as dermal or mucosal tissues.

In preferred embodiments, the subject method can be used in the treatment of human cancers expressing ErbB receptors. For example, Wnt antagonists are particularly useful in treating ErbB1 expressing cancers, such as ErbB1 expressing breast cancers.

Similarly, modulators of ErbB receptor activity may be useful in treating disease states involving Wnt. For example, modulators of ErbB receptor activity, which affect ErbB ligand binding, for example, could be of use in the treatment of diseases in which modulation of Wnt signalling is desired, such as in bone disorders, including bone cancer (in the case of Wnt-x), and in the treatment of infections such as bacterial, fungal, protozoan and viral infections; pain; diabetes; obesity; anorexia; bulimia; asthma; Parkinson's disease; acute heart failure; hypotension; hypertension; urinary retention; osteoporosis; angina pectoris; myocardial infarction; ulcers; asthma; allergies; benign prostatic hypertrophy; and psychotic and neurological disorders.

In certain embodiments, the subject agonists/antagonists are chosen on the basis of their selectively for the ErbB pathway. In particular embodiments, the Wnt antagonist is chosen for use because it is more selective for one ErbB isoform over the next, e.g., 10 fold, and more preferably at least 100 or even 1000 fold more selective for one ErbB pathway (e.g., ErbB1) over another.

In certain preferred embodiments, the Wnt inhibitors inhibit ErbB-mediated signal transduction with an ED₅₀ of 1 mM or less, more preferably of 1 microM or less, and even more preferably of 1 nM or less. The term “ED₅₀” means the dose of a drug, which produces 50% of its maximum response or effect. Alternatively, it means the dose that produces a pre-determined response in 50% of test subjects or preparations.

The compounds for use in the subject method may be conveniently formulated for administration with a biologically acceptable medium, such as water, buffered saline, polyol (for example, glycerol, propylene glycol, liquid polyethylene glycol and the like) or suitable mixtures thereof. The optimum concentration of the active ingredient(s) in the chosen medium can be determined empirically, according to procedures well known to medicinal chemists. As used herein, “biologically acceptable medium” includes any and all solvents, dispersion media, and the like which may be appropriate for the desired route of administration of the pharmaceutical preparation. The use of such media for pharmaceutically active substances is known in the art. Suitable vehicles and their formulation inclusive of other proteins are described, for example, in the book Remington's Pharmaceutical Sciences (Remington's Pharmaceutical Sciences. Mack Publishing Company, Easton, Pa., USA 1985). These vehicles include injectable “deposit formulations”.

In one aspect, the present invention therefore provides pharmaceutical preparations comprising, as an active ingredient, a Wnt antagonist for inhibition of ErbB-signalling activity (or an agonist for activating ErbB signalling activity), such as described herein. The subject treatments using Wnt agonists or antagonists can be effective for both human and animal subjects. Animal subjects to which the invention is applicable extend to both domestic animals and livestock, raised either as pets or for commercial purposes. Examples are dogs, cats, cattle, horses, sheep, pigs and goats.

The preparations of the present invention may be given orally, parenterally, topically, or rectally. They are of course given by forms suitable for each administration route. For example, they are administered in tablets or capsule form, by injection, inhalation, ointment, suppository, etc. Oral and topical administrations are preferred for small molecules. It is contemplated that the subject methods can be carried out using a variety of different small molecules, which can be readily Identified, e.g. by such drug screening assays as described herein. The above notwithstanding, in some embodiments, the methods and compositions of the present invention make use of antibodies or sFRP, or fragments thereof, which will typically be administered by suitable routes to maintain activity (for example, by avoiding protein degradation).

Thus also contemplated by the invention is the use of a Wnt agonist or antagonist as a pharmaceutical for the treatment of disorders or conditions dependent on ErbB signalling as well as the use of a Wnt antagonist or agonist in the manufacture of a medicament for the treatment of disorders of conditions dependent on ErbB.

In a further aspect of the invention, a method of diagnosing a patient in need of treatment with a Wnt agonist or antagonist is provided, comprising determining the presence of Wnt receptors in a sample from the patient, and detecting altered ErbB signalling in the sample relative to an unaffected individual. An increased level of ErbB signalling in a Wnt receptors (Fz)-containg sample correlates to a patient that can be treated with a Wnt antagonist. Conversely, a decreased level of ErbB signalling in a Wnt-receptor (Fz) containing sample correlates to a patient that can be treated with a Wnt agonist (including Wnt).

The invention will now be further described with reference to the following non-limiting Tables and Examples. Other embodiments of the invention will occur to those skilled in the art in the light of these.

### EXAMPLES

The examples are described for the purposes of illustration and are not intended to limit the scope of the invention.

Methods of molecular genetics, protein and peptide biochemistry and immunology referred to but not explicitly described in this disclosure and examples are reported in the scientific literature and are well known to those skilled in the art. For example, standard methods in genetic engineering are carried out essentially as described in Sambrook et al., Molecular Cloning: A laboratory manual, 2nd Ed., Cold Spring Harbor Laboratory Press, Cold Spring Harbor N.Y., 1989.

### Example 1

### Wnt1 and Wnt 5 Stimulate TCF Transcriptional Activity in HC 11 Mammary Cells

This example demonstrates that constitutive expression of Wnt1 and Wnt5a in HC 11 mammary cells, an immortalized cell line that has been used extensively to characterize the role of ErbB receptors in proliferation, differentiation and transformation (Taverna et al., 1991; Brandt et al., 2001), leads to elevated levels of TCF transcriptional activity as has previously been described in other systems.

HC11 mammary cells, were cultured in RPMI medium plus 10% FCS, containing EGF and insulin (Hynes et al., 1990) and maintained in this medium unless otherwise stated. HC11 cells were first co-transfected using SuperFectTM (Qiagen) with vectors encoding puromycin resistance and a TCF luciferase reporter (TopTK) (Brantjes et al., 2001). Cells were then selected in puro-containing medium. These TopTK expressing cells were used to prepare the Wnt-expressing HC11 cells lines. Two cell lines were made, one stably expressing Wnt1 and the other Wnt5a. The Wnt cDNAs are described at http://Aww.stanford.edu/˜musse/wntwindow.html.

To accomplish this, TopTK HC11 cells were infected with LNCX retroviruses (Miller A D, Rosman G J, 1989, Biotechniques 7: 980-990, which encode a gene for neomycin resistance and use the CMV promoter to drive Wnt1 or Wnt5a expression (Aberle et al 1997 EMBO J. 16: 3797-3804). Double drug resistant (puro/neo) pools of cells were selected and are referred to as Wnt1-HC11, Wnt5a-HC11 and Control (C)-HC11. These cells were either studied directly or used as a source of Wnt-containing conditioned medium. To prepare Wnt-containing CM, cells were grown overnight in medium lacking EGF and insulin then medium was removed and placed on cultures to be tested.

Microarray analyses were performed to determine Wnt and Fz expression in C-HC11 cells and to confirm expression of the introduced Wnt cDNA. RNA was prepared and analyzed according to the manufacturer's protocol on an Affymetrix GeneChip Murine Genome U74A array. Hybridization data were analyzed using the manufacturer's software (MAS4.0). Expression levels of Wnts and Fz are shown in arbritary units (Table 1).

TABLE 1

| | C-HC11 | Wnt1 cell line | Wnt5 cell line |
|---|---|---|---|
| Wnt1 | — | 20765 | — |
| Wnt4 | 3066 | — | 3252 |
| Wnt5a | — | 675 | 7630 |
| Wnt7b | 2891 | — | 3853 |
| Fz6 | 586 | | |
| Fz7 | 284 | | |
| Fz8 | 295 | | |

C-HC11 cells express mRNAs for Fz 6, -7 and -8, and Wnt 4 and -7b. High levels of Wnt1 and Wnt5a mRNA were detected in the respective transfected cell lines.

Elevation of cytosolic beta-catenin levels is considered a hallmark of beta-catenin's ability to bind and transcriptionally activate TCF (Bienz, 1998). Cytosolic fractions from each of the cell lines were therefore analysed for beta-catenin to determine whether TCF is activated. Briefly, cytosolic fractions were prepared following the method in Subcellular Fractonation, A Practical Approach, Edited by J M Graham and D. Rickwood, pp 21-22 and 50 micrograms were immunoblotted for beta-catenin and, as a loading control, tubulin, essentially as previously described (Lane et al., 2000). Beta-catenin and tubulin antibodies are commercially available (e.g., Transduction Laboratories, NeoMarkers). In comparison to C-HC11 cells, cytosolic fractions from Wnt1-and Wnt5a-HC11 cells have high levels of beta-catenin, whereas tubulin levels remained constant.

Whole cell lysates (WCL) were prepared by solubilizing cultures in NP40 extraction buffer (Lane et al., 2000) and used for immunoblotting using a TCF4 specific antibody (available commercially, e.g., Upstate Biotechnology); essentially as described previously (Lane et al., 2000). TCF4 was expressed in HC11 cells, Wnt1 and Wnt5a HC11 lines essentially at the same levels, suggesting that Wnts activate the prototypic beta-catenin/TCF pathway in HC11 cells, presumably via TCF4.

To examine short-term Wnt-induced signaling, C-HC11 cells were treated with conditioned medium (CM) harvested from cultures of Wnt1-and Wnt5a-HC11 cells. Wnt-containing conditioned medium (CM) was collected from cultures of Wnt1-or Wnt5a-HC11 cells grown overnight in medium without EGF and insulin, and added to C-HC11 cultures for 48 hours prior to assaying for luciferase. CM from both cultures caused a 4-5-fold increase in luciferase activity, confirming the biological activity of the secreted Wnt proteins in inducing the TCFpathway.

### Example 2

### ErbB1 is Activated in Wnt-Expressing HC11 Cells

ErbB1 is transactivated by many classes of membrane proteins (Carpenter, 1999; Gschwind et al., 2001). However, Wnt/VFz-mediated activation of ErbB1 has not been reported previously. Since ErbB1 tyrosine phosphorylation is an essential step in ErbB1 activation, we examined whether Wnt can activate ErbB1, by immunoprecipitating the receptor form Wnt/HC11 cells and performing a Westem analysis with phosphotyrosine specific antiserum. For these experiments HC11 cells were grown over night in medium without EGF. As a control for ErbB1 activation, C-HC 11 and Wnt-HC11 cells were treated for 10 minutes with 100 ng/ml EGF. An ErbB1 selective kinase inhibitor PKI166 (Traxler et al., 2001) was also used in some experiments. For this, the cells were pretreated 1 hr with 5 microM PKI 166, or left untreated, before preparing WCLs, essentially as described in Example 1.

ErbB1 was immunoprecipitated from 500 micrograms WCL and immunoblotted with a phosphotyrosine specific antibody (anti-phosphotyrosine AG10, Upstate Biotechnology) essentially as described previously (Lane et al., 2000). Membranes were stripped by placing them in 2% SDS, 62.5 mM Tris pH 6.8, 100 mM beta-mercaptoethanol at 60° C. for 30 min. Stripped membranes were reprobed for ErbB1 using a commercially available antibody (antibody 1005,Santa Cruz Biotechnology). Proteins were visualized with peroxidase-coupled secondary antibodies using the enhanced chemiluminescence detection system (Amersham Pharmacia Biotech, Little Chalfont, United Kingdom).

In contrast to ErbB1 from C-HC11 cells, which has low phosphotyrosine staining, ErbB1 was highly phosphorylated in Wnt1- and Wnt5a-HC11 cells. In HC11 cells, treatment with EGF (an ErbB1 activator) led to a strong increase in ErbB1 phosphorylation, but not in Wnt-expressing cells, suggesting that the receptor is already maximally activated in the latter. Neither ErbB2 nor ErbB3 was phosphorylated in control or Wnt-expressing HC11 cells (anti-ErbB2, (21N); Lane et al., 2000). Thus, the specific phosphorylation of ErbB1 indicates that ErbB1 is activated in Wnt expressing cells

Treatment of Wnt-HC11 cells with PKI166 inhibitor, an ErbB1 selective kinase inhibitor, for 1 hr decreased ErbB1's phosphotyrosine level by approximately 50%, suggesting that phosphorylation results from increased kinase activity and not from decreased phosphatase activity.

### Example 3

### MAPK is Activated in Wnt-Expressing HC11 Cells

The MAPK and PI3K pathways are major signaling cascades downstream of activated ErbB receptors (Olayioye et al., 2000). Antisera specific for the active, phosphorylated forms of ERK1/2 and PKB, the major kinases on the respective pathways, were used to probe for their activity. 50 micrograms of WCL were immunoblotted essentially as described above with the exception that a phospho-ERK1/2 antibody (commercially available, New England Biolabs) or a phosphoPKB antibody was used. Membranes were stripped and reprobed for ERK1/2 (anti-p44/42 ERK, New England Biolabs).

In Wnt1- and Wnt5a-HC11 cells, the basal level of phospho-ERK1/2, but not phospho-PKB, was elevated, compared to C-HC11 cells. In Wnt-1 cells, the degree of activation was equivalent to that observed in EGF-treated HC11 cells. The level of phospho-ERK1/2 was slightly less in Wnt5a-HC11 cells. Pretreatment of C-HC11 cells with PKI166 prevented EGF induction of MAPK activity. Furthermore, PKI166 treatment reduced the constitutive level of phospho-ERK1/2 to basal in the Wnt-expressing HC11 cells, suggesting that in these cells ErbB1 is responsible for activation of the MAPK pathway.

### Example 4

### Wnt Stimulates Intracellular Signaling Pathways in HC11 Cells

This Example shows that Wnt stimulates intracellular signalling pathways through ErbB1 in HC11 cells. Briefly, conditioned media (CM) from Wnt-HC11 cells prepared as described in Example 1 was tested for its effect on ErbB1 and MAPK activity, essentially as described above. Treatment of C-HC11 cells for 10 min with Wnt1- and Wnt5a-CM stimulated phospho-ERK1/2 levels 5-6-fold, whereas CM from C-HC11 cells had no effect. Moreover, in the presence of PKI166 (cells were pre-treated for 1 hour with PKI166), the ability of Wnt-containing CM to stimulate phospho-ERK1/2 levels was blocked, suggesting a direct involvement of ErbB1 in Wnt-induced ERK1/2 phosphorylation.

To rule out the potential involvement of ErbB1 ligands in mediating the effects of Wnt-containing CM, we used secreted Frizzled-related protein-1 (sFRP-1), which binds Wnt preventing it from binding Fz (Uren et al., 2000). CM from human embryonic kidney (HEK293) cells stably expressing the sFRP1 expression vector or control HEK 293 cells was premixed 1 hr with Wnt-containing CM (1:1) before adding to C-HC11 cultures for 10 min. Simultaneous addition of CM from sFRP1-producing 293 cells with Wnt-containing CM prevented Wnt1 and Wnt5a from increasing phospho-ERK1/2 levels, showing that sFRP1 blocks Wnt signalling through Fz to ErbB1. As a control, the cells were also treated for 10 minutes with EGF in the presence of sFRP1. EGF was able to induce ERK1/2 phosphorylation under these conditions, showing that sFRP1 only blocks Wnt mediated ErbB1 activation, but not ErbB1 ligand induced activation.

### Example 5

### Wnt-Mediated Activation of ErbB1 and ERK Requires Metalloproteinase Activity

The results of the Examples above demonstrate that in HC11 mammary cells Wnts have the ability to transactivate ErbB1 and to stimulate the MAPK pathway. A possible explanation for these results would be that Wnt binding to Fz causes an increase in the availability of ErbB1 activating ligands. HC11 cells, like the mammary gland (Troyer and Lee, 2001), express ErbB1 ligands. These include transforming growth factor-alpha (TGF-alpha) (Hynes et al., 1990), heparin-binding EGF (HB-EGF) and amphiregulin (AR). The ectodomains of these ligands are processed by metalloproteinases leading to shedding of soluble growth factors (Massague and Pandiella, 1993). These soluble peptides, in contrast to the membrane-bound forms appear to be responsible for most of the biological effects of the active receptor (Dong et al., 1999). In this Example, it was determined whether Wnt might increase the activity of specific metalloproteinases, which cleave these ligands. It has been demonstrated that ErbB1 transactivation by the GPCR-binding ligands endothelin and thrombin, results from metalloproteinase mediated proHB-EGF cleavage (Prenzel et al., 1999, Gschwind et al., 2000).

To test for a role of metalloproteinases in Wnt-expressing HC11 cells, two metalloproteinase inhibitors were used, namely phenanthroline (Calbiochem), a metal ion chelator, or CGS27023A, an enzymatic inhibitor (Wang et al., 1999). Cells were treated for 1 hour with phenathroline, CGS27023A, (both at 50μM) or DMSO carrier. ErbB1 was immunoprecipitated from 500 micrograms WCL and immunoblotted with a phosphotyrosine specific antibody, essentially as described above. Membranes were stripped and reprobed for ErbB1. ERK1/2 was probed from 50 micrograms of WCL, immunoblotted with a phospho-ERK1/2 antibody, essentially as described above. Membranes were stripped and reprobed for ERK1/2.

Treatment with either inhibitor led to a strong decrease in the level of phospho-ErbB1 and also lowered the level of phospho-ERK1/2 in the Wnt-expressing cells. In C-HC11 cells, ErbB1 and ERK1/2 display basal activity, both of which are further reduced in the presence of the metalloproteinase inhibitors, suggesting that autocrine activated ErbB1 is responsible for basal ERK1/2 activity. In the Wnt1 and Wnt5a-HC11 cells, this process appears to be enhanced.

The metalloproteinase inhibitors were next tested in the presence of Wnt-containing CM. C-HC11 cultures were pretreated 1 hr with metalloproteinase inhibitors before treatment with CM from C- or Wnt-HC11 cells for 10 minutes. Phospho-ERK1/2 levels were determined as above. Phenanthroline and CGS 27023A each blocked the ability of the Wnt proteins to stimulate ERK1/2 activity in C-HC11 cells. Similar results were obtained with HC11 cells constitutively expressing the Wnt proteins. Thus, Wnt-mediated activation of ErbB1 and ERK is dependent on a metalloproteinase activity.

### Example 6

### Wnt-Mediated Activation of ERK in HEK293 Cells is Blocked by a Monoclonal Antibody for ErbB1

The results of Example 5 suggest that Wnt/Fz-mediated activation of ErbB1 and the MAPK pathway occurs via metalloproteinase-induced ErbB1 ligand processing. An ErbB1 specific blocking antibody, which interferes with ligand binding to the extracellular domain of the receptor, was employed to confirm this supposition.

A monoclonal antibody, mAb528 (Commercially available—Santa Cruz), which interferes with ligand binding to the human ErbB1 receptor (Badache and Hynes, 2000) was used to evaluate Wnt signalling in human embryonic kidney (HEK293) cells, which endogenously express ErbB1. Methods are essentially as described above. HEK293 cultures were pretreated for 1 hour with 10 microgram/ml Ab528 and then for 10 minutes with CM from C- or Wnt-HC11 cells. As seen with HC11 cells, treatment of HEK-293 cells with Wnt1- and Wnt 5a-containing CM stimulated TCF transcriptional activity as well as MAPK activity. In the presence of mAb528 the ability of EGF, as well as Wnt-containing CM to stimulate MAPK activity was blocked, suggesting that Wnt binding to Fz increases the availability of ErbB1 ligands.

### Example 7

### HB-EGF is Expressed in Wnt-Expressing HC11 Cells

Quantitative real time PCR was carried out on HC11 cells or Wnt-expressing cells to determine which ErbB1 ligand might be involved in Wnt signalling to ERK. Briefly, mRNA was isolated from C- and Wnt-HC11 cells and the level of TGF-a, HB-EGF and AR was measured by real time PCR using specific oligos, essentially as previously described (Sorensen et al., 2000). The values are provided in Table 2 in arbritary units.

TABLE 2

| | TGF-alpha | HB-EGF | AR |
|---|---|---|---|
| HC11 | 3.66 | 1.48 | 35.8 |
| Wnt1 | 0 | 2.31 | 1.27 |
| Wnt5a | 2.69 | 2.42 | 32.3 |

The results revealed that TGF-alpha, HB-EGF and AR are expressed to similar levels in C-HC11 cells and in Wnt5a-HC11 cells, while Wnt1-HC11 cells have HB-EGF but no detectable TGF-alpha and reduced levels of AR. Thus, HB-EGF may mediate the effects of Wnts on ErbB1.

### Example 8

### Wnt1 and Wnt5a Stimulate Cyclin D1 Via ErbB1 Transactivation

Wnt target genes have have been identified in different biological systems. Relevant for this report is the finding that cyclin D1 has been reported to be a Wnt/β-catenin/TCF target gene in colon cancer cells (Tetsu and McCormick, 1999). Furthermore, in mammary tumors arising in MMTV-Wnt1 transgenic mice, there are high levels of cyclinD2, suggesting that cyclinD2 might be a Wnt1 target in the mammary gland (Yu et al., 2001).

Briefly, WCL was prepared from HC11, Wnt1- and Wnt5a-expressing HC11 cells and 50 μg immunoblotted essentially as described above using commercially available antibodies against cyclin D1 (NovaCastra) or cyclin D2 (SantaCruz). Both Wnt1- and Wnt5a-HC11 cells express higher levels of cyclin D1, in comparison to HC11 cells. In addition, Wnt1-HC11 cells but not C- or Wnt5a-HC11 cells express cyclin D2.

C-HC11 cells were also treated 6 hrs with CM from Wnt-HC11 cells. WCL were prepared and 50 μg was immunoblotted for Cyclin D1 or Cyclin D2. Similar to the results with the Wnt-HC11 cell lines, both Wnts (CM) stimulated Cyclin D1 expression, while only Wnt1 enhanced cyclin D2 levels.

To assess the contribution of Wnt-transactivated ErbB1 on cyclin D1 and D2 expression, C- and Wnt-HC11 cells were treated 6 hr with PKI166 or CGS27023A or EGF. PKI166 treatment reduced cyclin D1 to the control level in both Wnt1- and Wnt5a-HC11 cells, suggesting that ErbB1 activity is responsible for the increased cyclin D1 levels. These results are corroborated by the ability of EGF to stimulate cyclin D1 expression in control cells. In contrast, the ErbB1 inhibitor had no effect on cyclin D2 levels. Wnt-HC11 cells treated with the metalloproteinase inhibitor also displayed decreased expression of cyclin D1, but not cyclin D2. Thus, cyclin D1 and cyclin D2 are differentially controlled. Both Wnt1 and Wnt5a transactivate ErbB1, which results in increased expression of cyclin D1; in contrast cyclinD2 is only elevated in Wnt1 expressing cells and this is independent of ErbB1 activity.

In summary, the present inventors show that ErbB1 is transactivated by Wnt1 and Wnt5a in mammary epithelial cells. The ability of Wnt proteins to transactivate this receptor has not previously been described. Transactivated ErbB1 has a distinct effect in mammary cells, namely it is responsible for increasing cyclin D1 expression. In contrast, Wnt1 stimulates cyclin D2 expression in an ErbB1-independent manner.

The Examples above suggest that aberrant Wnt expression might contribute to breast cancer malignancy by increasing the activity of ErbB1. Thus, therapeutics designed to inhibit Wnt's ability to interact with its receptor can provide an additional means to down-regulate ErbB signaling thereby affecting the malignancy of cancers expressing ErbB, in particular breast cancers.

All publications cited above are hereby incorporated by reference, as if each were referred to individually.

1. A method for inhibiting EGF receptor signalling, said method comprising contacting a cell having EGF receptors and Frizzled (Fz) at the cell surface with a Wnt antagonist in a sufficient amount to reduce the EGF receptor signalling in said cell.

2. The method of claim 1, wherein said EGF receptor is ErbB1.

3. The method of claim 1, wherein said Wnt antagonist is an antagonist of Wnt1 or Wnt5a.

4. The method of the preceding claim, wherein the antagonist is an; antibody or fragment thereof, which specifically binds to Wnt.

5. The method of claims 1, wherein the antagonoist is an antibody or fragment thereof, which specifically binds to Fz.

6. The method of claims 1, wherein the antagonist is sFRP.

7. The method of as claimed in the preceding claims wherein said cell is an epithelial cell.

8. The method of as claimed in the preceding claims, wherein said cell is a solid tumor cell.

9. The method of claim 8, wherein said tumor cell is a breast cancer cell.

10. The use of a Wnt antagonist to inhibit EGF receptor signalling.

11. A method of screening for compounds effective in modulating Wnt-mediated ErbB receptor signalling, said method comprising:
(a) contacting a Wnt receptor (Fz) with Wnt in the presence of a candidate compound,
(b) detecting binding of Wnt or said candidate compound to said Wnt receptor and
(c) correlating the binding of said candidate compound to said Wnt receptor or a change in binding of Wnt to said Wnt receptor relative to when said candidate compound is absent with a potential ErbB modulator.

12. The method of claim 11, wherein said method is cell based.

13. The method of claim 12, further comprising detecting ErbB signalling.

14. The method of claim 13, wherein said ErbB signalling is detected by the presence of ERK activity, MARK activity, ErbB phosphotyrosine or cyclin D.

15. The method of claim 13, wherin said ErbB signalling is detected by the presence of a reporter gene product.

16. The method of claims 13, wherein said candidate compound inhibitis ErbB signalling.

17. A kit comprising:
(a) a Wnt, a Fz, and/or a cell expressing Wnt and/or Fz; and
(b) a means of detecting ErbB signalling.

18. The kit of claim 17, wherein said means of detecting ErbB signalling is an antibody.

19. The kit of claim as claim 18, wherein the antibody comprises a detectable tag or label.

20. A method for inhibiting ErbB signalling in a patient, comprising administering to the patient a composition comprising a Wnt antagonist in a sufficient amount to reduce the ErbB signalling in a cell of the patient.

21. The method of claim 20, wherein the antagonist is an antibody or a fragment thereof that specifically binds to Wnt.

22. The method of claim 20, wherein the antagonoist is an antibody or fragment thereof that specifically binds to Fz.

23. The method of claims 20, wherein the disorder is cancer.

24. The method of claim 23, wherein said cancer is breast or colon cancer.

25. The method of claim 24, wherein said cancer expresses ErbB1.

26. Use of a Wnt antagonist for the manufacture of a medicament for the treatment of ErbB expressing cancers.

27. A method of diagnosing a patient in need of treatment with a Wnt antagonist, said method comprising detecting ErbB receptor signalling.
